(19) United States
(12) Patent Application Publication
(10) Pub. No.: US 2021/0008101 A1
(43) Pub. Date: Jan. 14, 2021

(54) METHODS AND COMPOSITIONS FOR PREVENTING, REDUCING OR ERADICATING TOXICITY CAUSED BY ACETAMINOPHEN (APAP)

(71) Applicant: SINEW PHARMA INC., Taipei City (TW)

(72) Inventors: Oliver Yoa-Pu HU, Taipei City (TW); Tung-Yuan SHIH, Taipei City (TW); Cheng-Huei HSIONG, Taipei City (TW); Hsin-Tien HO, Taipei City (TW); Kai-Min CHU, New Taipei City (TW)

(73) Assignee: SINEW PHARMA INC., Taipei City (TW)

(21) Appl. No.: 16/969,864

(22) PCT Filed: Feb. 14, 2019
(86) PCT No.: PCT/CN2019/075052

§ 371 Date: Aug. 13, 2020

Related U.S. Application Data
(60) Provisional application No. 62/630,489, filed on Feb. 14, 2018.

(51) Int. Cl. A61K 31/78; A61K 31/765; A61K 31/7016; A61K 31/355; A61K 31/7004; A61K 45/06; A61P 39/02

(52) U.S. Cl. CPC A61K 31/78; A61K 31/765; A61K 31/7016; A61P 39/02; A61K 31/7004; A61K 45/06; A61K 31/355

(57) ABSTRACT

The present invention relates to methods and compositions for preventing, reducing or eradicating toxicity caused by acetaminophen (APAP). Specifically, the toxicity is nephrotoxicity and/or hepatotoxicity.

### RELATED APPLICATION

This application claims priority to U.S. Provisional Application No. 62/630,489, filed on Feb. 14, 2018, the content of which is hereby incorporated by reference in its entirety.

### TECHNOLOGY FIELD

The present invention relates to methods and compositions for preventing, reducing or eradicating toxicity caused by acetaminophen (APAP).

### BACKGROUND OF THE INVENTION

Acetaminophen (also known as Panadol) is also called paracetamol or N-acetyl-para-aminophenol (APAP) and is the most widely used pain-relieving and fever-reducing drug on the market. Each year, numerous cases of drug intoxication or suicide are reported due to improper use of APAP, and toxicity e.g. hepatotoxicity and nephrotoxicity caused by APAP is the main cause of severe diseases and death. A number of clinical studies have demonstrated that hepatotoxicity induced by APAP is preventable and early diagnosis along with real-time administration of the antidote N-acetylcysteine (NAC) can prevent the occurrence of hepatotoxicity.

Early detection of acetaminophen overdose is necessary because the best prognosis can be achieved if the antidote is given within 8 hours after poisoning. The early signs of drug intoxication include discomfort, nausea and vomiting. However, some patients may show no signs of intoxication at the early stage (stage 1) even if their blood concentrations of acetaminophen are at the poisoning levels and their abnormal liver function is apparently abnormal. The signs of hepatotoxicity, such as abdominal pain, persistent vomiting, jaundice, right upper quadrant pain, usually become apparent 24-48 hours after ingestion of a significant amount of acetaminophen (stage 2). Serum amintransferase usually starts to rise 16 hours after administration with clinical symptoms. Stage 3 usually occurs 3-4 days after administration and the degree of liver damage as well as prognosis can be well predicted at the time. The signs of hepatotoxicity progress from mild symptoms with elevated liver function values (AST>1,000 IU/L) to severe acute fulminant hepatitis accompanied by metabolic acidosis, jaundice, hyperglycemia, AST>1,000 IU/L, abnormal blood clotting and hepatic/brain lesions. Stage 4 will cause oliguria renal failure or death in severe cases.

Some patients with acetaminophen intoxication show only mild liver damage but with severe renal toxicity which is mainly caused by direct metabolism of APAP in P-450s (cytochrome P450s' CYPs) of the renal tubule. Nonetheless, acute renal failure may also result from hepatorenal syndrome caused by acute liver failure and the fraction excretion of Na (FeNa) can be used for differentiation primary renal damage (FeNa>1) from hepatorenal syndrome (FeNa>1). The calculation formula for FeNa is (Sodium urinary÷Creatinine urinary)÷(Sodium plasma÷Creatinine plasma)×100.

The peak concentration of acetaminophen in blood is achieved 1-2 hours after oral administration and a significant amount is eliminated by liver, more than 90% is conjugated to glucuronide and sulfate and form non-toxic metabolites and only less than 5% is eliminated by different CYPs, including CYP2E1, CYP1A2 and CYP3A4, and among which CYP2E1 and CYP1A2 are the major enzymes for metabolism. The metabolite produced by these enzymes, N-acetyl-p-benzoquinoneimine (NAPQI) is a very active electrophile. Under normal conditions, NAPQI will react immediately with glutathione in the cell and form non-toxic mercaptide. Overdose of acetaminophen makes the consumption rate of glutathione greater than its synthesis rate and when the glutathione level of the cell is lower than the normal range of 30%, NAPQI will bind to large molecules or nucleic acids containing cysteine and lead to liver damage. From histochemical stains, NAPQI will bind to the thiol group of cysteine and form a covalent bond in centrilobular areas before occurrence of liver cell necrosis.

Patients with liver disease, alcohol addiction or who are taking drugs which may induce the activity of P450 such as carbamazepine, ethanol, Isoniazid, Phenobarbital (may be other barbiturates), Phenytoin, Sulfinpyrazone, Sulfonylureas, Rifampin and Primidone are the susceptible groups of developing severe hepatotoxicity caused by APAP and may easily die if the patient also develops complications such as adult respiratory distress syndrome, cerebral edema, uncontrollable bleeding, infection or Multiple organ dysfunction syndrome (MODS). Take alcohol for example, alcohol is mainly eliminated by CYP2E1 of liver and its mechanism of APAP intoxication is divided into three stages: at the first stage alcohol competes the receptors for CYP2E1 with APAP in the liver and the concentration of NAPQI will reduce during the stage, at the second stage alcohol prolongs the half-life of CYP2E1 from 7 hours to 37 hours which increases the level of CYP2E1 in the liver and the concentration of NAPQ1 will slowly increase during this stage, and at the third stage, during alcohol withdrawal, more CYP2E1 is found in the liver to eliminate acetaminophen and consequently the toxic metabolites of acetaminophen increases significantly and lead to liver damage. Recent studies have shown that diallyl sulfide can effectively prevent hepatotoxicity caused by acetaminophen in mice and further demonstrated diallyl sulfide can inhibit the activity of CYP2E1. It is speculated that the protection mechanism of diallyl sulfide against hepatotoxicity induced by acetaminophen is by inhibition of the production of the intermediate NAPQI from acetaminophen. Previous studies have suggested by inhibition the consumption of reduced glutathione in liver cells, oxidation activation, mitochondrial dysfunction and DNA damage caused by NAPQI can be reduced and subsequently minimize liver damage induced by acetaminophen. For example, Panax notoginseng, adenosine and its derivatives adenosine monophosphate, adenosine diphosphate and adenosine triphosphate can prevent liver damage induced by acetaminophen through this protection mechanism.

There is still a need to solve the toxicity of APAP and to provide an effective analgesic approach and an APAP preparation with reduced or free of toxicity.

### BRIEF DESCRIPTION OF THE DRAWINGS

The embodiment is shown below to illustrate the present invention. It should be understood, however, that the present invention is not limited to the preferred embodiment shown. In the drawing:

FIG. 1 shows histological examination results that (FIG. 1A) Normal structure of kidney in normal control group; (FIG. 1B) Stained section of kidney of acetaminophen 1 g/kg group. Severe necrosis, congestion and extravasation of red blood cells were seen; (FIG. 1C) Stained section of kidney of acetaminophen 1 g/kg and NAC group. Mild to moderate degenerations were seen; and (FIG. 1D) Stained section of kidney of acetaminophen 1 g/kg and test compound group, a combination of Eudragit S100, sucralose and Luteolin. Structures of kidney were similar to the normal control group. Haematoxylin and eosin. Magnification 200×.

### BRIEF SUMMARY OF THE INVENTION

In one aspect, the present invention provides a method for preventing, reducing or reducing or eradicating toxicity caused by acetaminophen (APAP) or its derivative, comprising administering to a subject in need thereof a compound selected from the group consisting of Eudragit S100, Pluronic F68, Nariagenin, Kaempferol, Mannitol, Sucralose, Luteolin, menthol, polyethylene glycol sorbitan monolaurate (Tween 20), Microcrystalline cellulose, Brij 35, Saccharin, Cremophor RH40, Crospovidone, Sodium starch glycolate, Eudragit S100, Croscarmellose sodium, Low-substituted hydroxypropyl cellulos, Pregelatinized starch, Dextrates NF hydrated, Citric acid, Cremophor EL, Aerosil 200, Myrj 52, Sorbic acid, Lemon oil, Hydroxypropyl cellulose, Sorbitol, Acesulfame potassium, Hydroxypropyl methylcellulose, Lactose monohydrate, Maltodextrin, Brij 58, Brij 76, Tween 80, Tween 40, PEG 400, PEG 4000, PEG 8000, Span 60, Sodium benzoate, Hydroxy ethylmethylcellulose, Methylcellulose, Span 80, Sodium cyclamate, Glyceryl behenate, Oxide red, Glycerin monostearate, Copovidone K28, Starch acetate, Magnesium stearate, Sodium lauryl sulfate, Providone K30, PEG 2000 and any combination thereof, in an amount effective in preventing, reducing or eradicating toxicity caused by APAP.

In one aspect, the present invention provides a method for preventing, reducing or reducing or eradicating toxicity caused by acetaminophen (APAP) or its derivative, comprising administering to a subject in need thereof a compound selected from the group consisting of Eudragit S100, Pluronic F68, Nariagenin, Kaempferol, Mannitol, Sucralose, Luteolin and any combination thereof, in an amount effective in preventing, reducing or eradicating toxicity caused by APAP.

In some embodiments, the compound includes a first compound selected from the group consisting of Eudragit S100, Pluronic F68, Nariagenin, Kaempferol and any combination thereof. In some embodiments, the compound includes a second compound selected from the group consisting of Mannitol, Sucralose, Luteolin and any combination thereof.

In one aspect, the present invention provides a method for preventing, reducing or reducing or eradicating toxicity caused by acetaminophen (APAP) or its derivative, comprising administering to a subject in need thereof a first compound selected from the group consisting of Eudragit S100, Pluronic F68, Nariagenin, Kaempferol and any combination thereof, in an amount effective in preventing, reducing or eradicating toxicity caused by APAP.

In some embodiments, the toxicity is nephrotoxicity and/or hepatotoxicity.

In some embodiments, the first compound includes a combination of Eudragit S100, Pluronic F68 and Nariagenin.

In some embodiments, the method of the present invention further comprises administering to the subject a second compound selected from the group consisting of Mannitol, Sucralose, Luteolin and any combination thereof.

In some embodiments, the first compound and the second compound administered to the subject is a combination selected from the group consisting of:

(i) a combination of Eudragit S100 and sucralose;

(ii) a combination of Pluronic F68 and sucralose;

(iii) a combination of Eudragit S100 and mannitol;

(iv) a combination of Pluronic F68 and mannitol;

(v) a combination of Eudragit S100, sucralose and Luteolin;

(vi) a combination of Kaempferol, Mannitol and Sucralose; and

(v) a combination of Nariagenin, Mannitol and Sucralose.

Also provided is use of a compound (a first compound and/or a second compound and/or any combination thereof) as described herein for manufacturing a medicament (e.g.an antidote) for preventing, reducing or eradicating toxicity caused by acetaminophen (APAP) or its derivative (e.g. as an acetaminophen toxicity preventer or inhibitor). Further provided is a composition for preventing, reducing or eradicating toxicity caused by acetaminophen (APAP) or its derivative comprising a compound (a first compound and/or a second compound and/or any combination thereof) as described herein. In particular, a first compound is used optionally in combination with a second compound as described herein.

In another aspect, the present invention provides a method for preventing, reducing or eradicating nephrotoxicity caused by acetaminophen (APAP), comprising administering to a subject in need thereof a compound (a first compound and/or a second compound and/or any combination thereof), in an amount effective in preventing, reducing or eradicating nephrotoxicity caused by APAP.

In some embodiments, the compound includes a first compound selected from the group consisting of Eudragit S100, Pluronic F68, Nariagenin, Kaempferol and any combination thereof. In some embodiments, the compound includes a second compound selected from the group consisting of a second compound selected from the group consisting of Mannitol, Sucralose, Luteolin and any combination thereof.

Also provided is use of a compound (a first compound and/or a second compound and/or any combination thereof) as described herein for manufacturing a medicament (an antidote) for preventing, reducing or eradicating nephrotoxicity caused by acetaminophen (APAP) or its derivative (e.g. as an acetaminophen toxicity preventer or inhibitor). Further provided is a composition for preventing, reducing or eradicating nephrotoxicity caused by acetaminophen (APAP) comprising a compound (a first compound and/or a second compound and/or any combination thereof) as described herein.

In a further aspect, the present invention provides a method for administering APAP to treat a condition treatable by APAP in combination with one or more antidote compounds as described herein. Also provided is a combination of APAP in combination with one or more antidote compounds as described herein.

The details of one or more embodiments of the invention are set forth in the description below. Other features or advantages of the present invention will be apparent from the following detailed description of several embodiments, and also from the appending claims.

### DETAILED DESCRIPTION OF THE INVENTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The term “comprise” or “comprising” is generally used in the sense of include/including which means permitting the presence of one or more features, ingredients or components. The term “comprise” or “comprising” encompasses the term “consists” or “consisting of.”

As used herein, the term “about” or “approximately” refers to a degree of acceptable deviation that will be understood by persons of ordinary skill in the art, which may vary to some extent depending on the context in which it is used. In general, “about” or “approximately” may mean a numeric value having a range of ±10% around the cited value.

The present invention discloses that one or more of the compounds as described herein have the effects in preventing, reducing or eradicating toxicity caused by caused by acetaminophen (APAP). Thus, the present invention provides use of one or more of the compounds as described herein for manufacturing a medicament (e.g. an antidote) for preventing, reducing or eradicating toxicity caused by APAP (e.g. as an acetaminophen toxicity preventer or inhibitor). The present invention also provides a method for preventing, reducing or eradicating toxicity caused by APAP by administering to a subject in need an effective amount of one or more of the compounds as described herein. The present invention further provides a composition comprising one or more of the compounds as described herein for use in preventing, reducing or eradicating toxicity caused by caused by APAP.

As used herein, the term “acetaminophen (APAP)” is intended to include the chemical derivatives of the acetaminophen structure having equivalent pharmaceutical effect.

As used herein, the toxicity caused by APAP can include nephrotoxicity and/or hepatotoxicity. Nephrotoxicity and hepatotoxicity can include both functional toxicity and histological changes in kidney and liver. Injuries in liver may include injuries, damages or loss of hepatic cells or tissues, leading to abnormal liver functions or contents of liver proteins. In some embodiments, the liver injuries as described herein are acute liver injuries which mean liver injuries of relatively rapid onset e.g. less than 12 week, particularly less than 6 weeks duration from time of onset of symptoms. In some embodiments, patients with acute liver injuries are with no background of chronic hepatic diseases. Liver function can be determined by many routine assays, such as alanine aminotransferase (ALT) or aspartate transaminase (AST) analysis for liver function. Injuries in kidney may include injuries, damages or loss of renal cells or tissues, leading to abnormal renal functions. Such renal injuries may be identified, for example, by a decrease in glomerular filtration rate, a reduction in urine output, an increase in serum creatinine, an increase in serum cystatin C, etc. In some embodiments, the renal injuries as described herein are acute renal injuries, which may mean an abrupt or rapid decline in renal filtration function, for example, within 14 days, preferably within 7 days, more preferably within 72 hours, and still more preferably within 48 hours. Renal function can be determined by many routine assays, such as creatinine or blood urea nitrogen (BUN) measurement.

An increased level of an index or condition of toxicity may be used as an indicator of induction or occurrence of the toxicity (a toxic state) which is compared with reference to a control (or normal) level thereof. As used herein, a “normal level” or “control level” is meant to describe a value within an acceptable range of values that one of ordinary skill in the art and/or a medical professional e.g. a doctor would expect a healthy individual or population of similar physical characteristics and medical history to have. A “decreased” level of an index or condition of toxicity can be used as an indicator of reduction or removal of toxicity when compared with that of a corresponding toxic state. Especially, when a decreased level of an index or condition of toxicity comes close to or even becomes lower than a normal level or control level, the toxicity can be considered “eradicated.”

As used herein, the toxicity such as nephrotoxicity and/or hepatotoxicity can be caused by overdose of APAP. Overdose can refer to administration of a dose greater than a useful or standard dose that is an effective dose approved by a drug regulatory authority such as Food & Drug Administration or prescribed by a physician for treatment or prevention of a diseases condition or relief of symptoms thereof. For example, paracetamol tablets are the currently APAP drugs approved in the market for oral administration, the standard dose of which is 500 mg to 1 g paracetamol taken every 4-6 hours as required, up to a maximum of 4 g daily, for a human adult. Overdose of APAP can mean a dose greater than a useful or standard dose of APAP, for example, by 5%, 10%, 20%, 30%, 50%, 75%, 100% or more.

As used herein, the term “treating” refers to the therapeutic measures to a disease or the symptoms or conditions of a disease, which include but are not limited to applying or administering one or more active agents to a subject suffering from the disease or the symptoms or conditions of the disease or exacerbation of the disease. The purpose of the therapeutic measures is to treat, cure, mitigate, relieve, alter, remedy, ameliorate, improve, or affect the disease, the symptoms or conditions of the disease, disability caused by the disease, or exacerbation of the disease.

As used herein, the term “individual” or “subject” includes human or non-human animals, in particular mammal, for example, companion animals (such as dogs, cats and the like), farm animals (such as cattle, sheep, pigs, horses, etc.), or laboratory animals (such as rats, mice, guinea pigs, etc.).

As used herein, the term “effective amount” refers to the amount of an active ingredient achieving desired biological efficacy or therapeutic effects in a subject being treated, for example, preventing or reducing toxicity in liver or kidney in the subject receiving overdose of APAP.

For the purpose of transport and uptake, an effective amount of an active ingredient according to the present invention may be formulated with a pharmaceutically acceptable carrier to form a suitable form of a pharmaceutical composition. According to the routes of administration, the pharmaceutical composition of the present invention preferably comprise from about 0.1% to about 100% by weight of the active ingredient, based on the total weight of the composition. As used herein, the term “pharmaceutically acceptable” means that the carrier is compatible with the active ingredient of the composition (and does not affect the effect of the active ingredient), and, preferably, the carrier may stabilize the active ingredient and is safe for the subjects being treated. The carrier may be a diluent, a vehicle, an excipient, or a medium for the active ingredient. The composition of the present invention can provide the effect of rapid, continued, or delayed release of the active ingredient after administration to the patient. According to the present invention, the form of said composition may be tablets, pills, powder, lozenges, packets, troches, elixers, suspensions, lotions, solutions, syrups, soft and hard gelatin capsules, suppositories, sterilized injection fluid, and packaged powder.

The composition of the present invention may be delivered via any physiologically acceptable route, such as oral, parenteral (such as intramuscular, intravenous, subcutaneous, and intraperitoneal), transdermal, suppository, and intranasal methods. Regarding parenteral administration, it is preferably used in the form of a sterile water solution, which may comprise other substances, such as salts or glucose sufficient to make the solution isotonic to blood. Preparation of an appropriate parenteral composition under sterile conditions may be accomplished with standard pharmacological techniques well known to persons skilled in the art, and no extra creative labor is required.

The present invention also provides a method for administering APAP to treat a condition (e.g. pain) treatable by APAP characterized in that APAP is administered in combination with one or more antidote compound(s) as described herein, simultaneously or sequentially. In certain embodiments, APAP can be administered in an overdose with reduced or free of toxicity when compared with administration of the same dose of APAP alone without one or more antidote compound(s) as described herein. For example, APAP can be administered higher than 4 g daily for a human adult, for example, 5 g or more daily, 6 g or more daily, 7 g or more daily, 8 g or more daily, 9 g or more daily or 10 g or more daily.

The present invention further provides a combination of APAP and one or more antidote compound(s) as described herein. In certain embodiments, the combination includes an amount of APAP higher than a normal dose for a single dose (e.g. 500 mg for a human adult), for example, 600 mg or higher, 700 mg or higher, 800 mg or higher, 900 mg or higher, 1,000 mg or higher.

The present invention is further illustrated by the following examples, which are provided for the purpose of demonstration rather than limitation.

### EXAMPLES

1. Materials and Methods

1.1 Animal

Male Sprague-Dawley rats (weighing 220-300 g) were obtained from Professor Dr. Gonzalez of the American Country Health Research Institute (USA), introduces three male and four female mice, pairs voluntarily reproduces. All of the experiments were performed in adherence with the National Institutes of Health Guidelines for the treatment of animals. All of the mice were maintained in a room with air/humidity control and a 12-h light/dark cycle, and allowed access to food and water ad libitum throughout the experiment.

1.2 APAP-Induced Toxicity in an Animal Model

In the single dose study of the antidote effect of APAP metabolic enzyme inhibition on APAP-induced nephrotoxicity, rats were randomly divided into 3 treatment groups: (1) a normal control group (NC, n=5), in which normal SD-rats were orally administrated with saline four times a day for multiple doses at a volume of 1 mL/kg; (2) an APAP control group (APAP, n=5), in which normal SD-rats were orally administrated with APAP in saline at dose of 1 g/kg (1,000 mg/kg) for single oral dose administration; (3) an positive control group (NAC, n=5), in which normal SD-rats were orally administrated with APAP and N-acetylcysteine (NAC) in saline at dose of 1 g/kg and 140 mg/kg for single oral dose administration; (4) the various selected antidote test groups (each groups, n=5), in which normal SD-rats were orally administered with APAP in saline at dose of 1,000 mg/kg and e various antidote agents each at dose of less than or equivalent to 200 mg per 60 kg person body weight of single oral dose administration. In addition, according to US FDA Guidance for Industry “Estimating the Maximum Safe Starting Dose in Initial Clinical Trials for Therapeutics in Adult Healthy Volunteers”, the HED (human equivalent dose) for a human adult of 60 kg can be calculated as follows: HED=animal dose (1,000 mg/kg in rats)×human body weight (60 kg)/6.2=9.677g (about 10 g).

1.3 Blood Sampling

At the end of the treatment, the animals were sacrificed under diethyl ether anesthesia. Blood was collected from the hearts of the mice into heparin tubes, and plasma was separated by centrifugation at 13,000×g for 5 min at 4° C. Aliquots of plasma were transferred to Eppendorf tubes and stored at −80° C. until analysis.

1.4 Biochemical Investigation of Serum for Kidney Function

Serum creatinine and BUN levels were measured using the commercial kits (BEN) (Biochemical Enterprise, Milano, Italy) CR280 and BK151 in an automatic biochemical analyzer (ChemWell, Palm City, Fla.) at 340 nm and 510 nm, respectively.

1.5 Hepatic AST and ALT Determination

Plasma enzyme activities (aspartate aminotransferase [AST] and alanine aminotransferase [ALT]) were determined at 37° C. by using a Synchron LXi 725 (Beckman Instruments, Palo Alto, Calif. USA) with kits provided by the manufacturer.

1.6 Histological Examinations

The kidney groups were fixed in 10% neutral formalin for 48-72 h. Then, tissues were trimmed and processed for a routine histopathological examination. Tissues were embedded into paraffin wax and 4-5 mm thick sections were cut. All tissue sections were stained with Haematoxylin and Eosin (H&E) and then examined under alight microscope (OlympusBX51, Tokyo, Japan). Renal lesions in rats were assessed according to Zhang et al. (2008) and graded into five categories by utilizing a scale of 0-5, where 0 =normal histology and 1 =tubular epithelial cell degeneration, without significant necrosis/apoptosis; 2-5:<25%, <50%, <75% and ≥75% of the tubules showed tubular epithelial cell necrosis/apoptosis, respectively, accompanied by other concomitant alterations.

1.7 Statistical Analysis

All data are represented in mean ± standard deviation (SD) and the results are calculated using ANOVA to determine the significance. Statistical Package of the Social Science program (Version 13, SPSS Inc.) is used for calculations followed by post hoc test to examine the least significant difference for multiple comparisons so as to confirm the significant differences between groups and the average difference between groups was significant p<0.05.

2. Results

The body weights, relative liver and kidney weights of the experimental animals were measured at the end of study. There were no statistical significant differences compared with the control animals.

Toxicity including nephrotoxicity and hepatotoxicity were successfully induced by oral administration of APAP at a dose of 1,000 mg/kg once daily for single dose. In the toxicity control group, the serum BUN (87.8 ±6.6 mg/dL) and creatinine (1.13±0.17 mg/dL) were significantly increased when compared with the normal control group (18.7±2.5 and 0.29±0.04 mg/dL, respectively) (p<0.005), indicating renal damages has occurred in the toxicity control group; and the plasma AST level (591.0±59.2 IU/L) and the plasma ALT level (382.3±32.1 IU/L) were significantly increased when compared with the normal control group (190.0+27.2 IU/L and 49.9±4.7 IU/L, respectively), indicating liver damage has occurred in the toxicity control group. A first group of compounds including Eudragit S100, Pluronic F68, Nariagenin and Kaempferol, and a second group of compounds including mannitol, sucralose and luteolin were tested for their antidote activity against toxicity caused by APAP. Table 1 shows the results.

Table 1 shows that the tested compounds are effective as antidotes against toxicity (nephrotoxicity and hepatotoxicity) caused by APAP.

| | Creatinine (mg/dL) | BUN (mg/dL) | AST (IU/L) | ALT (IU/L) |
|---|---|---|---|---|
| | nephrotoxicity | | hepatotoxicity | |
| Normal control | 0.29 ± 0.04 | 18.7 ± 2.5 | 190.0 ± 27.2 | 49.9 ± 4.7 |
| AAP toxicity | 1.13 ± 0.17 | 87.8 ± 6.6 | 591.0 ± 59.2 | 382.3 ± 32.1 |
| Positive control: NAC | 0.64 ± 0.05 | 48.4 ± 9.8*** | 423.7 ± 38.1** | 206.7 ± 20.1*** |
| 1st Cpd | Creatinine (mg/dL) | BUN (mg/dL) | AST (IU/L) | ALT (IU/L) |
| Eudragit S100 | 0.32 ± 0.07*** | 24.4 ± 2.8*** | 237.2 ± 20.0*** | 58.0 ± 5.4*** |
| Pluronic F68 | 0.37 ± 0.13*** | 29.1 ± 3.4*** | 253.2 ± 22.1*** | 80.1 ± 13.4*** |
| Nariagenin | 0.32 ± 0.03*** | 36.0 ± 7.8*** | 231.1 ± 39.0*** | 63.1 ± 11.6*** |
| Kaempferol (high dose) | 0.38 ± 0.09*** | 31.5 ±7.6*** | 332.2 ± 27.1*** | 82.2 ± 17.6*** |
| Kaempferol (low dose) | 0.70 ± 0.09*** | 58.8 ± 7.0*** | 395.6 ± 32.6*** | 179.4 ± 12.9*** |
| Nariagenin + Pluronic | | | | |
| F68 + Eudragit S100 | 0.22 ± 0.04*** | 20.3 ± 4.1*** | 188.0 ± 18.3*** | 48.2 ± 5.6*** |
| | Creatinine (mg/dL) | BUN (mg/dL) | AST (IU/L) | ALT (IU/L) |
| 2nd Cpd | nephrotoxicity | | hepatotoxicity | |
| Sucralose (S) | 0.40 ± 0.15*** | 33.6 ± 2.7*** | 288.4 ± 33.8*** | 82.8 ± 8.4*** |
| Mannitol (M) | 0.56 ± 0.13*** | 41.0 ± 2.6*** | 365.7 ± 35.5*** | 91.9 ± 8.4*** |
| Luteolin (high) | 0.25 ± 0.07*** | 23.7 ± 4.4*** | 223.8 ± 15.5*** | 57.1 ± 14.7*** |
| Luteolin (low) | 0.43 ± 0.08*** | 31.5 ± 7.3*** | 279.1 ± 35.1*** | 78.9 ± 15.7*** |
| M + S | 0.35 ± 0.08*** | 28.2 ± 4.2*** | 242.7 ± 27.7*** | 57.5 ± 11.6*** |
| | Creatinine (mg/dL) | BUN (mg/dL) | AST (IU/L) | ALT (IU/L) |
| 1st Cpd + 2nd Cpd | nephrotoxicity | | hepatotoxicity | |
| Kaempferol + M + S | 0.18 ± 0.03*** | 16.2 ± 2.8*** | 187.5 ± 12.5*** | 50.5 ± 5.5*** |
| Nariagenin + M + S | 0.19 ± 0.03*** | 15.0 ± 3.0*** | 174.9 ± 4.6*** | 49.7 ± 2.4*** |
| Eudragit S100 + Luteolin + S | 0.19 ± 0.03*** | 16.7 ± 2.8*** | 192.7 ± 10.5*** | 49.7 ± 5.4*** |
| Eudragit S100 + S | 0.28 ± 0.04*** | 22.9 ± 5.3*** | 226.2 ± 27.1*** | 58.4 ± 6.4*** |
| Pluronic F68 + S | 0.32 ± 0.06*** | 24.5 ± 2.0*** | 269.0 ± 8.5*** | 71.0 ± 6.0*** |
| Eudragit S100 + M | 0.34 ± 0.09*** | 25.5 ± 2.2*** | 225.0 ± 23.2*** | 58.1 ± 12.0*** |
| Pluronic F68 + M | 0.42 ± 0.07*** | 35.5 ± 5.5*** | 282.2 ± 20.7*** | 78.1 ± 11.5*** |

As shown in Table 1, the tested compounds, including the first group of compounds (Eudragit S100, Pluronic F68, Nariagenin, Kaempferol) and the second group of compounds (mannitol, sucralose, luteolin) are effective in reducing or eradicating toxicity (nephrotoxicity and hepatotoxicity) caused by APAP. In particular, a combination of Nariagenin, Pluronic F68 and Eudragit S100 exhibits superior antidote activities. Further, one of the first group of compounds in combination with one or more of the second group of compounds exhibits enhanced (synergistic) antidote activities, better than a first compound or a second compound alone.

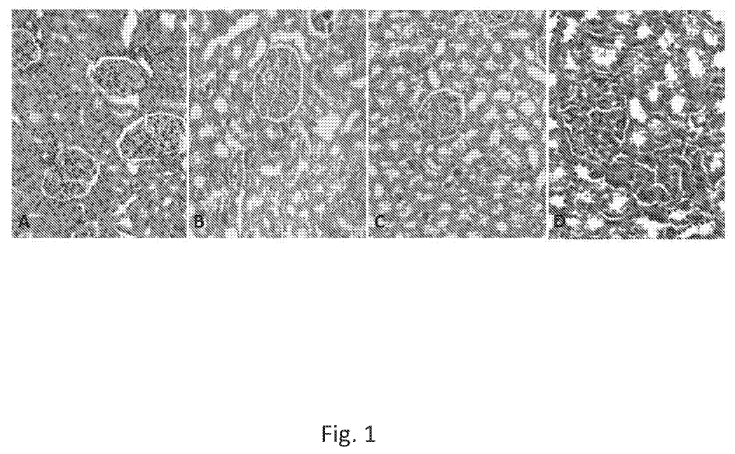

In addition, as shown in the histological examination results of FIG. 1, severe renal lesions including necrosis, congestion and extravasation of red blood cells were observed in the APAP overdose group (FIG. 1B). In contrast, such renal lesions were reduced to mild to moderate level in the NAC group (FIG. 1C) and greatly reduced to close to the normal control state in the group receiving the test compounds of the present invention (FIG. 1D).

Given the above, the compounds as described herein can be used as antidotes to prevent, reduce or eradicate toxicity (nephrotoxicity and hepatotoxicity) caused by APAP. These compounds belong to pharmaceutically acceptable excipients or natural plant phenolic compounds, which are all considered safe through animal experiments. The present invention therefore provides methods and compositions for preventing, reducing or eradicating toxicity caused by acetaminophen (APAP) using one or more of such antidote compounds. The present invention also provide a method for administering APAP to treat a condition treatable by APAP in combination with one or more of such antidote compounds, with reduced or eradicated toxicity when compared with administration of APAP alone. A combination of APAP and one or more of such antidote compounds is also provided.

1. A method for preventing, reducing or eradicating toxicity caused by acetaminophen (APAP) or its derivative, comprising:
administering to a subject in need thereof a first compound selected from the group consisting of Eudragit S100, Pluronic F68, Nariagenin, Kaempferol, Mannitol, Sucralose, Luteolin, menthol, polyethylene glycol sorbitan monolaurate (Tween 20), Microcrystalline cellulose, Brij 35, Saccharin, Cremophor RH40, Crospovidone, Sodium starch glycolate, Eudragit S100, Croscarmellose sodium, Low-substituted hydroxypropyl cellulos, Pregelatinized starch, Dextrates NF hydrated, Citric acid, Cremophor EL, Aerosil 200, Myrj 52, Sorbic acid, Lemon oil, Hydroxypropyl cellulose, Sorbitol, Acesulfame potassium, Hydroxypropyl methylcellulose, Lactose monohydrate, Maltodextrin, Brij 58, Brij 76, Tween 80, Tween 40, PEG 400, PEG 4000, PEG 8000, Span 60, Sodium benzoate, Hydroxy ethylmethylcellulose, Methylcellulose, Span 80, Sodium cyclamate, Glyceryl behenate, Oxide red, Glycerin monostearate, Copovidone K28, Starch acetate, Magnesium stearate, Sodium lauryl sulfate, Providone K30, PEG 2000 and any combination thereof, in an amount effective in preventing, reducing or eradicating toxicity caused by APAP or its derivative.

2. The method of claim 1, wherein the first compound is selected from the group consisting of Eudragit S100, Pluronic F68, Nariagenin, Kaempferol and any combination thereof.

3. The method of claim 1, wherein the toxicity is nephrotoxicity and/or hepatotoxicity.

4. The method of claim 2, wherein the first compound comprises a combination of Eudragit S100, Pluronic F68 and Nariagenin,

5. The method of claim 2, further comprising administering to the subject a second compound selected from the group consisting of Mannitol, Sucralose, Luteolin and any combination thereof.

6. The method of claim 5, wherein the first compound and the second compound administered to the subject is a combination selected from the group consisting of
(i) a combination of Eudragit S100 and sucralose;
(ii) a combination of Pluronic F68 and sucralose;
(iii) a combination of Eudragit S100 and mannitol;
(iv) a combination of Pluronic F68 and mannitol;
(v) a combination of Eudragit S100, sucralose and Luteolin;
(vi) a combination of Kaempferol, Mannitol and Sucralose; and
(v) a combination of Nariagenin, Mannitol and Sucralose.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. A method for preventing, reducing or eradicating nephrotoxicity caused by acetaminophen (APAP) or its derivative, comprising:
administering to a subject in need thereof a first compound selected from the group consisting of Eudragit S100, Pluronic F68, Nariagenin, Kaempferol, Mannitol, Sucralose, Luteolin, menthol, polyethylene glycol sorbitan monolaurate (Tween 20), Microcrystalline cellulose, Brij 35, Saccharin, Cremophor RH40, Crospovidone, Sodium starch glycolate, Eudragit S100, Croscarmellose sodium, Low-substituted hydroxypropyl cellulos, Pregelatinized starch, Dextrates NF hydrated, Citric acid, Cremophor EL, Aerosil 200, Myrj 52, Sorbic acid, Lemon oil, Hydroxypropyl cellulose, Sorbitol, Acesulfame potassium, Hydroxypropyl methylcellulose, Lactose monohydrate, Maltodextrin, Brij 58, Brij 76, Tween 80, Tween 40, PEG 400, PEG 4000, PEG 8000, Span 60, Sodium benzoate, Hydroxy ethylmethylcellulose, Methylcellulose, Span 80, Sodium cyclamate, Glyceryl behenate, Oxide red, Glycerin monostearate, Copovidone K28, Starch acetate, Magnesium stearate, Sodium lauryl sulfate, Providone K30, PEG 2000 and any combination thereof, in an amount effective in preventing, reducing or eradicating toxicity caused by APAP or its derivative.

20. The method of claim 19, wherein the first compound is selected from the group consisting of Eudragit S100, Pluronic F68, Nariagenin, Kaempferol and any combination thereof,
said method optionally further comprising administering to the subject a second compound selected from the group consisting Mannitol, Sucralose, Luteolin and any combination thereof, in an amount effective in reducing or eradicating nephrotoxicity caused by APAP or its derivative.

21. The method of claim 20, wherein the first compound comprises a combination of Eudragit S100, Pluronic F68 and Nariagenin.

22. The method of claim 20, wherein the second compound is administered and comprises mannitol and sucralose.

23. The method of claim 20, wherein a combination of the first compound and the second compound is administered to the subject.

24. The method of claim 23, wherein the combination of the first compound and the second compound is selected from the group consisting of
(i) a combination of Eudragit S100 and sucralose;
(ii) a combination of Pluronic F68 and sucralose;
(iii) a combination of Eudragit S100 and mannitol;
(iv) a combination of Pluronic F68 and mannitol;
(v) a combination of Eudragit S100, sucralose and Luteolin;
(vi) a combination of Kaempferol, Mannitol and Sucralose; and
(v) a combination of Nariagenin, Mannitol and Sucralose.

25. (canceled)

26. (canceled)

27. (canceled)

28. (canceled)

29. A method for administering APAP to treat a condition treatable by APAP in a subject in need, comprising:
administering a therapeutically effective amount of APAP or its derivative in combination with a first compound selected from the group consisting of Eudragit S100, Pluronic F68, Nariagenin, Kaempferol, Mannitol, Sucralose, Luteolin, menthol, polyethylene glycol sorbitan monolaurate (Tween 20), Microcrystalline cellulose, Brij 35, Saccharin, Cremophor RH40, Crospovidone, Sodium starch glycolate, Eudragit S100, Croscarmellose sodium, Low-substituted hydroxypropyl cellulos, Pregelatinized starch, Dextrates NF hydrated, Citric acid, Cremophor EL, Aerosil 200, Myrj 52, Sorbic acid, Lemon oil, Hydroxypropyl cellulose, Sorbitol, Acesulfame potassium, Hydroxypropyl methylcellulose, Lactose monohydrate, Maltodextrin, Brij 58, Brij 76, Tween 80, Tween 40, PEG 400, PEG 4000, PEG 8000, Span 60, Sodium benzoate, Hydroxy ethylmethylcellulose, Methylcellulose, Span 80, Sodium cyclamate, Glyceryl behenate, Oxide red, Glycerin monostearate, Copovidone K28, Starch acetate, Magnesium stearate, Sodium lauryl sulfate, Providone K30, PEG 2000 and any combination thereof, in an amount effective in preventing, reducing or eradicating toxicity caused by APAP or its derivative.

30. The method of claim 29, wherein the first compound is selected from the group consisting of Eudragit S100, Pluronic P68, Nariagenin, Kaempferol and any combination thereof,
said method optionally further comprising administering to the subject a second compound selected from the group consisting Mannitol, Sucralose, Luteolin and any combination thereof, in an amount effective in preventing, reducing or eradicating toxicity caused by APAP or its derivative.

31. A combination comprising:
a therapeutically effective amount of APAP or its derivative; and
a first compound selected from the group consisting of Eudragit S100, Pluronic F68, Nariagenin, Kaempferol, Mannitol, Sucralose, Luteolin, menthol, polyethylene glycol sorbitan monolaurate (Tween 20), Microcrystalline cellulose, Brij 35, Saccharin, Cremophor RH40, Crospovidone, Sodium starch glycolate, Eudragit S100, Croscarmellose sodium, Low-substituted hydroxypropyl cellulos, Pregelatinized starch, Dextrates NF hydrated, Citric acid, Cremophor EL, Aerosil 200, Myrj 52, Sorbic acid, Lemon oil, Hydroxypropyl cellulose, Sorbitol, Acesulfame potassium, Hydroxypropyl methylcellulose, Lactose monohydrate, Maltodextrin, Brij 58, Brij 76, Tween 80, Tween 40, PEG 400, PEG 4000, PEG 8000, Span 60, Sodium benzoate, Hydroxy ethylmethylcellulose, Methylcellulose, Span 80, Sodium cyclamate, Glyceryl behenate, Oxide red, Glycerin monostearate, Copovidone K28, Starch acetate, Magnesium stearate, Sodium lauryl sulfate, Providone K30, PEG 2000 and any combination thereof, in an amount effective in preventing, reducing or eradicating toxicity caused by APAP or its derivative.

32. The combination of claim 31, wherein the first compound is selected from the group consisting of Eudragit S100, Pluronic F68, Nariagenin, Kaempferol and any combination thereof,
said combination optionally further comprising a second compound selected from the group consisting Mannitol, Sucralose, Luteolin and any combination thereof, in an amount effective in preventing, reducing or eradicating toxicity caused by APAP or its derivative.

33. The method of claim 29, wherein the APAP or its derivative is administered in an amount greater than a normal dose.

34. The combination of claim 31, wherein the APAP or its derivative is present in an amount greater than a normal dose.
